(19) United States
(12) Patent Application Publication
(10) Pub. No.: US 2015/0011818 A1
(43) Pub. Date: Jan. 8, 2015

(54) TUMOR VESSEL EMBOLIZING AGENT AND METHOD OF EMBOLIZING TUMOR VESSEL

(71) Applicant: ACADEMIA SINICA, Taipei (TW)

(72) Inventors: Yeu-Kuang Hwu, Taipei (TW); Chia-Chi Chien, Taipei (TW)

(21) Appl. No.: 14/324,183

(22) Filed: Jul. 5, 2014

(30) Foreign Application Priority Data
Jul. 4, 2013 (TW) 102123932

(51) Int. Cl. A61K 33/24; A61N 5/10; A61K 41/00

(52) U.S. Cl. CPC A61K 33/24; A61K 41/0038; A61N 5/10; A61N 2005/1098
USPC 600/1; 424/649; 424/489

(57) ABSTRACT

The present disclosure provides a tumor vessel embolizing agent, including: unmodified gold nanoparticles; and a pharmaceutically acceptable medium. The present disclosure also provides a method of embolizing tumor vessel, including administrating gold nanoparticles as a tumor vessel embolizing agent into a subject to accumulate the gold nanoparticles at a tumor in the subject and to embolize a vessel of the tumor.

### CROSS REFERENCE TO RELATED APPLICATIONS

This application claims priority of Taiwan Patent Application No. 102123932, filed on Jul. 4, 2013, the entirety of which is incorporated by reference herein.

### BACKGROUND

1. Technical Field

The disclosure relates to a tumor vessel embolizing agent, and in particular to a tumor vessel embolizing agent using nanoparticles.

2. Description of the Related Art

A vessel tumor embolism of the head, neck and central nervous system has become an important treatment method in addition to a surgical operation. This embolism may decreases the rates of outbreak and death, and may assist to remove a portion of a tumor at the same time. For tumors which cannot be treated with a surgical operation, an embolism may be the main treatment method. The embolism is usually performed by vessel transportation. However, the embolus may also be directly injected into the tumor by penetration injection. The vessel transportation is usually performed by invasively penetrating a micro catheter into an artery (for example, the inguinal femoral artery, carotid artery, etc.) and then the micro catheter is led to the tumor to inject the embolus, embolizing the blood supply to the tumor. This embolus may be permanent or temporary. For example, this embolus may be liquid (ethanol, acrylic acid, Onyx) or particles (poly(vinyl alcohol), Gelfoam).

Gold nanoparticles (Au-NPs) have been used in a variety of nanotechnology applications, such as bio-sensing, biological imaging, and nanoscale treatment. Au-NPs play an important role in the biomedical fields such as health, diagnosis, and fighting malignant diseases such as cancer. Au-NPs are small in size and have Enhanced Permeability and Retention Effect (EPR) in tumor parts, and are able to selectively agglomerate in cancer tissues. Therefore, Au-NPs are suitable as drug-delivery carriers or radiotherapy enhancers.

It is desirable to provide a vessel embolizing agent which has specificity to a tumor and may block a tumor vessel to inhibit the growth of the tumor, and provide real-time monitoring as well.

### SUMMARY

The present disclosure provides a tumor vessel embolizing agent, including: unmodified gold nanoparticles; and a pharmaceutically acceptable medium.

The present disclosure also provides a method of embolizing tumor vessel, including administrating gold nanoparticles as a tumor vessel embolizing agent into a subject to accumulate the gold nanoparticles at a tumor in the subject and to embolize a vessel of the tumor.

A detailed description is given in the following embodiments with reference to the accompanying drawings.

### BRIEF DESCRIPTION OF THE DRAWINGS

The disclosure may be more fully understood by reading the subsequent detailed description and examples with references made to the accompanying drawings, wherein:

FIG. 1 is an X-ray image (exposure time: 100 milliseconds) of gold nanoparticles accumulated and embolized in a tumor vessel after arterially injecting the gold nanoparticles into mice, wherein the circular part is the scope of the tumor.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

The present disclosure utilizes unmodified gold nanoparticles as a tumor vessel embolizing agent and injects the unmodified gold nanoparticles into a tumor vessel in a subject. The subject is irradiated by an X-ray source to accumulate and embolize the gold nanoparticles with high absorption contrast at the tumor vessel. The X-ray image of the gold nanoparticles is then used to observe the tumor.

The X-ray source may be a synchrotron radiation X-ray source, a medical X-ray source, or a laboratory X-ray source. X-ray source has wavelength ranging from about 3.09×10⁻¹ to 6.1×10⁻⁵ nm and energy ranging from about 4 keV-20 MeV. X-ray may overcome the inadequate penetration of photons in vivo, and is able to efficiently stimulate the nanoparticles administrated in the subject. In addition, X-ray source irradiation may be performed for less than about 1 second (60 minutes), preferably less than about 200 milliseconds (5 minutes). The effective penetration depth of the subject irradiated by the X-ray source may be about 30 cm from the surface to the deep tissue. Since the high-energy X-ray source adopted in the present disclosure has a high penetration ability in vivo, tumor cells in vivo may be monitored in real-time by X-ray imaging of the present disclosure, saving the need for sample slicing from living subjects as conventional medical imaging requires.

The present disclosure is suitable to embolize the tumor vessel in a subject. In one embodiment, subjects may be mammals, birds, amphibians, reptiles, fish, insects, or other appropriate multicellular animals.

In some embodiments, the tumor may include, but is not limited to, an epithelial tumor, brain tumor, melanoma tumor, lymphatic tumor, plasmacytoma, carneus tumor, ganglioglioma, thymic tumor, or a tumor in the oral cavity, esophagus, digestive system, respiratory system, bone, joint, soft tissue, skin, breast, reproductive system, urinary system, eye, eye socket, brain, other nervous system, endocrine system, lymph, bone marrow and etc.

In one embodiment, methods of administering the unmodified gold nanoparticles to a subject may include, but are not limited to, intravenous injection, arterial injection, lymphatic injection, or local organ injection. In one embodiment, the gold nanoparticles are injected into a tumor upstream artery.

The gold nanoparticles used herein are unmodified. In one embodiment, the gold nanoparticles in the present disclosure are grown by a synchrotron radiation method. This method may include providing gold ion-containing precursor solution such as HAuCl₄.3H₂O solution and adjusting the pH value of this precursor solution to make this precursor solution basic to prevent aggregation and size non-uniformity. For example, the pH value of the precursor solution may be adjusted to about 8-11. Then this precursor solution is irradiated by synchrotron radiation X-ray (such as 4-30 keV X-ray from a BL01A beamline) to transform the precursor into gold nanoparticles. The size of the gold nanoparticles may be adjusted according to the irradiation time. The longer the irradiation time, the smaller the size of the resulting gold nanoparticles. In some embodiments, the gold nanoparticles may range from 1 nm-100 μm, preferably from 1-50 nm. The gold nanoparticles are relatively inert, non-toxic and harmless to the subject.

In some embodiments, the gold nanoparticles may be combined with a pharmaceutically acceptable medium such as a solvent, dispersant or isotonic agent. In some embodiments, the pharmaceutically acceptable medium may include water, physiological saline, sugar, gel, porous matrix, preservative or a combination thereof. In some embodiments, the pharmaceutically acceptable medium is water. In one embodiment, the concentration of the gold nanoparticles may range from about 1-1000 mg/ml, preferably from about 1-300 mg/ml. For example, the concentration of the gold nanoparticles may be about 190 mg/ml. The injection dose of the gold nanoparticles may range from about 0.0001-100 g/kg, preferably from about 0.0001-2 g/kg. For example, the injection dose of the gold nanoparticles may be about 0.19 g/kg.

### EXAMPLE

### Example 1

### In Vivo X-ray Image of Gold Nanoparticles Accumulated at Tumor Vessel

The mice used in this example were BALB/c mice (purchased from the National Laboratory Animal Center, Taiwan) approved by the Academia Sinica Institutional Animal Care and Utilization Committee (AS IACUC). All mice were housed in individual cages (five per cage) and kept at 24±2° C. with a humidity of 40%-70% and a 12-hour light/dark cycle.

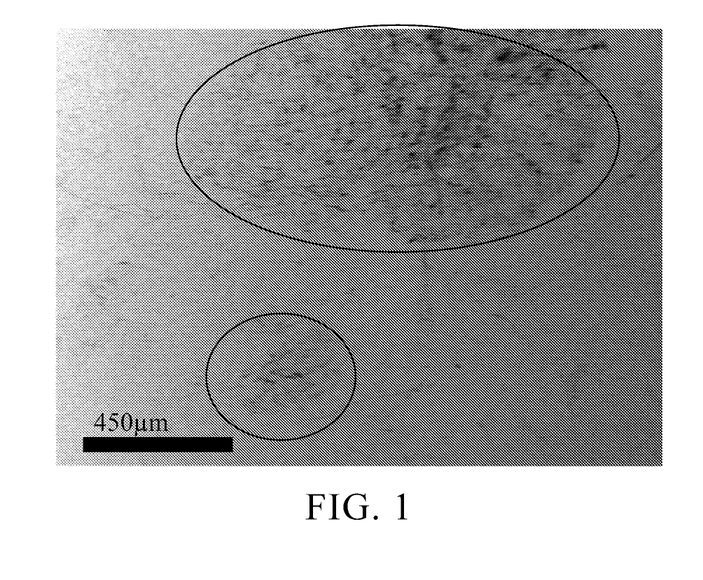

4-5 week-old mice were anesthetized by intramuscular injection of 10 μl of Zoletil 50 (50 mg/kg; Virbac Laboratories, Carros, France). A PE-08 catheter was inserted into a carotid artery of each of the mice (about 20-25 g of weight). Then 1000 μl, 50 mM of the above gold nanoparticle-containing contrast dye was injected into a late-stage tumor (16 days) in the mice from the carotid artery through the PE-08 catheter (PE-08 catheters, BB31695, Scientific Commodities, Inc., I.D.: 0.2 mm, O.D.: 0.36 mm). The injection rate of the contrast dye was 1 μl/s. During the development, the mice were kept under anesthesia using 1% isoflurene in oxygen. The image was an X-ray image taken 5 minutes after the injection of the mouse from its carotid artery. The exposure time was 100 milliseconds and the wavelength of the synchrotron radiation X-ray ranged from about 3.09×10⁻¹-4.13×10⁻² nm nm. The energy of the synchrotron radiation X-ray ranged from about 4 keV-30 keV. The X-ray source had a dose of less than about 100 Gy. The result is shown in FIG. 1, which is the X-ray image of the tumor vessel in the mice with the gold nanoparticles accumulated and embolized in the tumor vessel.

Although some embodiments of the present disclosure and their advantages have been described in detail, it should be understood that various changes, substitutions and alterations can be made herein without departing from the spirit and scope of the disclosure as defined by the appended claims. For example, it will be readily understood by those skilled in the art that many of the features, functions, processes, and materials described herein may be varied while remaining within the scope of the present disclosure. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A tumor vessel embolizing agent, comprising:
unmodified gold nanoparticles; and
a pharmaceutically acceptable medium.

2. The embolizing agent as claimed in claim 1, wherein the tumor vessel embolizing agent is injected into a tumor artery of a subject.

3. The embolizing agent as claimed in claim 2, wherein the subject comprises mammals, birds, amphibians, reptiles, fish, insects, and/or other appropriate multicellular animals.

4. The embolizing agent as claimed in claim 1, wherein the tumor comprises epithelial tumor, brain tumor, melanoma tumor, lymphatic tumor, plasmacytoma, carneus tumor, ganglioglioma, thymic tumor, or tumor at oral cavity, esophagus, digestive system, respiratory system, bone, joint, soft tissue, skin, breast, reproductive system, urinary system, eye, eye socket, brain, other nervous system, endocrine system, lymph, and bone marrow.

5. The embolizing agent as claimed in claim 1, wherein a diameter of the nanoparticles ranges from 1 nm to 100 μm.

6. The embolizing agent as claimed in claim 1, wherein the pharmaceutically acceptable medium comprises a solvent, dispersant or isotonic agent.

7. A method of embolizing tumor vessel, comprising administrating gold nanoparticles as a tumor vessel embolizing agent into a subject to accumulate the gold nanoparticles at a tumor in the subject and to embolize a vessel of the tumor.

8. The method as claimed in claim 7, further comprising irradiating the tumor of the subject by an X-ray source to monitor in real-time the tumor vessel embolizing agent at the tumor of the subject.

9. The method as claimed in claim 8, wherein the X-ray source comprises a synchrotron radiation X-ray source, a medical X-ray source, or a laboratory X-ray source.

10. The method as claimed in claim 8, wherein the X-ray source has a dose of less than about 100 Gy.

11. The method as claimed in claim 8, wherein the X-ray source has a dose between about 1 Gy and 30 Gy.

12. The method as claimed in claim 8, wherein the irradiation is performed for less than about 1 minute (60 minutes).

13. The method as claimed in claim 8, wherein the irradiation is performed for less than about 200 milliseconds (5 minutes).
